(19) United States
(12) Patent Application Publication
(10) Pub. No.: US 2003/0235856 A1
(43) Pub. Date: Dec. 25, 2003

(54) Oligonucleotide for detection and identification of mycobacteria

(76) Inventors: Cheol Min Kim, Pusan Metropolitan (KR); Hee Kyung Park, Seoul (KR); Hyun Jung Jang, Pusan Metropolitan (KR)

Correspondence Address: CANTOR COLBURN, LLP, 55 GRIFFIN ROAD SOUTH, BLOOMFIELD, CT, 06002

(21) Appl. No.: 10/448,914

(22) Filed: May 30, 2003

Related U.S. Application Data
(62) Division of application No. 09/980,052, filed on Nov. 28, 2001.
Application No. 09/980,052 is a 371 of international application No. PCT/KR00/00477, filed on May 16, 2000.

(30) Foreign Application Priority Data
May 29, 1999 (KR) 1999-19631
May 29, 1999 (KR) 1999/19632
May 29, 1999 (KR) 1999/19633
May 29, 1999 (KR) 1999/19634
May 29, 1999 (KR) 1999/19635
Apr. 7, 2000 (KR) 2000/18189

(51) Int. Cl. C12Q 1/68; C07H 21/04

(52) U.S. Cl. 435/6; 536/23.7

(57) ABSTRACT

The present invention relates to oligonucleotides of probes or primers for detection or identification of Mycobacterium. In the claimed invention, oligonucleotide sequences of ITS (Internal Transcribed Spacer Region) from M. fortuitum, M. chelonae, M. abscessus, M. vaccae, M. flavescens, M. asiaticum, M. porcinum, M. acapulcensis and M. diernhoferi have been identified. Using these ITS sequences, PCR primers or hybridization probes for detection or identification of Mycobacterium have been developed and presented as seq ID: 10 to seq ID: 241.

### CROSS REFERENCE TO RELATED APPLICATIONS

This application is a divisional application of U.S. patent application Ser. No. 09/980,052, filed on Nov. 28, 2001, which claims priority to PCT/KR00/00477, filed on May 16, 2000 and is herein incorporated by reference in its entirety.

### BACKGROUND OF THE INVENTION

1. Field of the Invention

The present invention relates to oligonucleotides that can be used for detection and identification of mycobacteria. Most particularly, the present invention identifies the nucleotide sequence of ITS (Internal Transcribed Spacer region) of non-tuberculosis mycobacteria, Mycobacterium fortuitum, Mycobacterium chelonae, Mycobacterium abscessus, Mycobacterium vaccae, Mycobacterium flavescens, Mycobacterium asiaticum, Mycobacterium porcinum, Mycobacterium acapulcensis and Mycobacterium diernhoferi, and using the nucleotide sequences, it provides oligonucleotide primers or probes used for detection and identification of mycobacteria.

2. Description of the Related Art

Even though the number of patients of tuberculosis has steadily decreased in these days, about 8 million patients have come out and about 3 million patients died of tuberculosis in a year. Moreover, in underdeveloped countries, inadequate treatment and lack of drugs for tuberculosis increase chronic carriers of drug-resistant bacteria. In 1980's, spread of AIDS has increased patients of tuberculosis even in advanced countries. In this condition, it is expected that about twelve million patents of tuberculosis would newly come out in the year 2000 (J. P. Natain, M. C. Raviglione, and A. Kochi, Tubercle and Lung Disease, 73; 311-321, 1992; Murray CJL. And Lopez AD. The global burden of disease. Global burden of disease and injury series. Vol. 1. Cambridge, Mass: Harvard University Press, 1996, p349-350; Global Tuberculosis Programme: Anti-tuberculosis drug resistance, WHO Report 1997: World Health Organization. 1997).

In 1950's, it was reported that non-tuberculosis mycobacteria (NTM) has been able to cause diseases in human. After the report that M. avium complex (MAC) would bring about systemic disease in the patients of AIDS in 1980, non-tuberculosis mycobacteria have taken an interest. Diseases caused by non-tuberculosis mycobacteria are similar to tuberculosis in clinical condition and general pathological view. Non-tuberculosis mycobacteria are distributed in a wide range of living environment, and it is difficult to judge whether they have pathogenicity or not in clinical test sample. Further, since they have resistance to a number of drugs for tuberculosis, the infection is hard to treat and the recurrence rate is high. The infection of non-tuberculosis mycobacteria should be treated by other means than for tuberculosis, and therefore, accurate and fast method of detecting and identifying non-tuberculosis mycobacteria is required. The accurate and fast method of detecting and identifying both TB complex and NTM is also needed for effective treatment and management of tuberculosis.

Many a method has been developed to diagnose mycobacterial infection and to detect and identify mycobacteria strains. Among them, the following methods are used at present:

The first is a microbiological method, that is, smearing, staining and culturing test. However, this method is not suitable for mycobacteria, since they have long generation term and need long culturing time. Further such pathogenic microorganism as mycobacteria is dangerous to infect the personnel in culture room.

The second is a PCR (Polymerase Chain Reaction) method. It is highly sensitive and specific to the mycobacteria and very useful to detect mycobacteria which have a long culturing time. Especially, it does not require a culturing process but uses a small amount of DNA to be amplified, therefore, only a small amount of pathogens in test sample is enough to detect and identify mycobacteria. Many a PCR process has been introduced with different target DNAs each other, and IS6110 and 16S rRNA are often used as the target (Bauer J, Andersen AB, Kremer K, and Miorner H, Usefulness of spoligotyping to discriminate IS6110 low-copy-number Mycobacterium tuberculosis complex strains cultured in Denmark, 1999, J. Clin Microbiol, 37: 2602-2606; Troesch, A., H. Nguyen, C. G. Miyada, S. Desvarenne, T. R. Gingeras, P. M. Kaplan, P. Cros and C. Mabilat. 1999, Mycobacterium species identification and rifampin resistance testing with high-density DNA probe arrays, J. Clin. Microbiol. 37: 49-55);

The third is a physico-chemical process, in which lipid component in mycobacteria has been detected by HPLC, GC or mass spectrophotometer. This method is very specific but requires expensive equipments;

The fourth is a method of detecting mycobacteria composition by serological method. This method uses a coagulation reaction of latex particles or blood corpuscles adsorbed with antibody to mycobacterial antigen or enzyme-linked immunological method in which enzyme is linked with antibody. It is, however, very sensitive only to be proceeded within a limited place. Further, it is difficult for this method to distinguish present infection from previous infection;

The next method to detect mycobacteria consists of infecting mycobacteria with mycobacteriophage L5 inserted with luciferase gene, and inspecting luminescence by luciferin in medium (W. R. Jacobs, R. G. Barletta, R. Udani, J. Chan, G. Kalkut, G. Sosne, T. Kieser, G. J. Sarkis, G. F. Hatful, and B. R. Bloom. 1993, Science 260: 819-822); and

The last is a method of detecting and identifying mycobacteria by hybridization of oligonucleotide (A. Troesch, H. Nguyen, C. G. Miyada, S. Desvarenne, T. R. Gingeras, P. M. Kaplan, P. Cros and C. Mabilat. 1999. J. Clin. Microbiol. 37: 49-55).

Besides Mycobacterium avium complex (MAC) described above, M. fortuitum, M. chelonae complex, M. terrae and M. vaccae are also known as non-tuberculosis mycobacteria. Among them, M. chelonae complex are classified into M. chelonae and M. abscessus, and there is no means to distinguish one from the other at present.

### SUMMARY OF THE INVENTION

To solve the problems in the prior method of detection and identification of mycobacteria, it is an objective of the present invention to provide specific oligonucleotides as probes or primers for PCR which can be used to detect mycobacteria, to distinguish TB complex from NTM, and to identify species of mycobacteria with an accuracy and effectiveness.

To accomplish the above objective, the present invention provides a DNA of ITS (Internal Transcribed Spacer region) of Mycobacterium fortuitum, Mycobacterium chelonae, Mycobacterium abscessus, Mycobacterium vaccae, Mycobacterium flavescens, Mycobacterium asiaticum, Mycobacterium porcinum, Mycobacterium acapulcensis and Mycobacterium diernhoferi genes set forth in SEQ ID Nos: 1 to 9.

Further, the present invention provides, as a primer for PCR or a probe for hybridization, an oligonucleotide for detection of mycobacteria set in forth in one of SEQ ID Nos: 10 to 14;

an oligonucleotide for distinction of TB complex from NTB among mycobacteria set in forth in one of SEQ ID NOs: 15 to 23;

an oligonucleotide for detection and identification of MAC (Mycobacterium avium and Mycobacterium intracellulare) set in forth in one of SEQ ID NOs: 24 to 27;

an oligonucleotide for detection and identification of Mycobacterium fortuitum set forth in one of SEQ ID NOs: 28 to 38;

an oligonucleotide for detection and identification of Mycobacterium chelonae set forth in one of SEQ ID NOs: 39 to 46;

an oligonucleotide for detection and identification of Mycobacterium abscessus set forth in one of SEQ ID NOs: 47 to 52;

an oligonucleotide for detection and identification of Mycobacterium vaccac set forth in one of SEQ ID NOs: 53 to 64;

an oligonucleotide for detection and identification of Mycobacterium flavescens set forth in one of SEQ ID NOs: 65 to 72;

an oligonucleotide for detection and identification of Mycobacterium gordonae set forth in one of SEQ ID NOs: 73 to 77;

an oligonucleotide for detection and identification of Mycobacterium terrae set forth in one of SEQ ID NOs: 78 to 100;

an oligonucleotide for detection and identification of Mycobacterium scrofulaceum set forth in one of SEQ ID NOs: 101 to 108;

an oligonucleotide for detection and identification of Mycobacterium kansasii set forth in one of SEQ ID NOs: 109 to 112;

an oligonucleotide for detection and identification of Mycobacterium szulgai set forth in one of SEQ ID NOs: 113 to 116;

an oligonucleotide for detection and identification of Mycobacterium marinum and Mycobacterium ulcerans set in forth in one of SEQ ID NOs: 117 to 119;

an oligonucleotide for detection and identification of Mycobacterium gastri set forth in one of SEQ ID NOs: 120 to 123;

an oligonucleotide for detection and identification of Mycobacterium xenopi set forth in one of SEQ ID NOs: 124 to 133;

an oligonucleotide for detection and identification of Mycobacterium genavense set forth in one of SEQ ID NOs: 134 to 141;

an oligonucleotide for detection and identification of Mycobacterium malmoense set forth in one of SEQ ID NOs: 142 to 146;

an oligonucleotide for detection and identification of Mycobacterium simiae set forth in one of SEQ ID NOs: 147 to 153;

an oligonucleotide for detection and identification of Mycobacterium smegmatis set forth in one of SEQ ID NOs: 154 to 165;

an oligonucleotide for detection and identification of Mycobacterium shimoidei set forth in one of SEQ ID NOs: 166 to 172;

an oligonucleotide for detection and identification of Mycobacterium habana set forth in one of SEQ ID NOs: 173 to 180;

an oligonucleotide for detection and identification of Mycobacterium farcinogen set forth in one of SEQ ID NOs: 181 to 189;

an oligonucleotide for detection and identification of Mycobacterium asiaticum set forth in one of SEQ ID NOs: 190 to 193;

an oligonucleotide for detection and identification of Mycobacterium porcinum set forth in one of SEQ ID NOs: 194 to 205;

an oligonucleotide for detection and identification of Mycobacterium acapulcensis set forth in one of SEQ ID NOs: 206 to 215;

an oligonucleotide for detection and identification of Mycobacterium diernhoferi set forth in one of SEQ ID NOs: 216 to 227;

an oligonucleotide for detection and identification of Mycobacterium paratuberculosis set in forth in one of SEQ ID NOs: 228 to 240; and

an oligonucleotide for detection of Mycobacteria sp. set forth in SEQ ID NO: 241.

In the prior method of detecting and identifying mycobacteria using PCR, only one or two strains can be detected. According to the present invention, however, almost all of mycobacteria strains can be detected and identified, since primers and probes of the present invention have been designed from DNA sequences of ITS of mycobacteria. ITS has more polymorphic region than 16S rRNA has and ITS also has conserved region, therefore, it is highly effective as a target DNA for distinction of genotype (Gurtler, V., A. Stanisich, 1996, New approaches to typing and identification of bacteria using the 16S-23S rDNA spacer region. Microbiol. 142: 3-16).

The inventors identified DNA sequences of ITS of non-tuberculosis mycobacteria whose DNA had not yet been sequenced, such as Mycobacterium fortuitum, Mycobacterium chelonae, Mycobacterium abscessus, Mycobacterium vaccae, Mycobacterium flavescens, Mycobacterium asiaticum, Mycobacterium porcinum, Mycobacterium acapulcensis and Mycobacterium diernhoferi. Using the DNA sequences, oligonucleotides for primers or probes have been designed for detecting and identifying the mycobacteria. Further, referring to the information on DNA sequence of other mycobacteria disclosed in GenBank, and analyzing the information with multi-alignment and blast, distinctive regions of polymorphism were selected to design oligonucleotides for primers or probes to detect and identify mycobacteria. The oligonucleotides have been confirmed to detect and identify mycobacteria by specific hybridization and amplification with species-specific and genus-specific primers of PCR.

That is, the oligonucleotide probes of the present invention, attached to solid substrate, are hybridized only with nucleotide sequence in ITS of specific mycobacteria, and therefore, they can detect and identify the specific mycobacteria sensitively. Further, the oligonucleotide primers of identical nucleotide sequence with the above probes can also detect and identify the specific mycobacteria by amplification in PCR. Using the oligonucleotide primers or probes made from ITS of mycobacteria, it is possible to detect mycobacteria, distinguish TB complex from NTM, and identify mycobacteria species accurately and effectively.

### BRIEF DESCRIPTION OF THE DRAWINGS

The above and other objects, features, and advantages of the present invention will be apparent from the following detailed description of the preferred embodiment of the invention in conjunction with the accompanying drawings, in which:

FIG. 1 is a schematic diagram showing ITS of mycobacteria and primers used to amplify the ITS by PCR;

FIG. 2 is a photograph showing the result of electrophoresis after PCR using several mycobacteria strains and a pair of primers for ITS amplification including a part of 16S rRNA and 23S rRNA of mycobacteria;

FIG. 3 is a photograph showing the result of electrophoresis after PCR using several mycobacteria strains and a pair of primers (ITSF and MYC2) for detecting mycobacteria;

FIG. 4 is a schematic diagram of multiplex PCR for detecting mycobacteria and simultaneously distinguishing TB complex from NTM;

FIG. 5 is a photograph showing the result of electrophoresis after multiplex PCR using a pair of primers (ITSF and MYC2) for detecting mycobacteria and a pair of primers (MTB2 and MYC2) for distinguishing TB complex from NTM;

FIG. 6 is a photograph showing the result of electrophoresis after PCR using each mycobacteria and each pair of species-specific primers designed from nucleotide sequence of polymorphic region of each NTM; and

FIG. 7 is a photograph showing the result of electrophoresis after PCR using several mycobacteria and each pair of species-specific primers designed from nucleotide sequence of polymorphic region of each NTM.

### DETAILED DESCRIPTION OF THE INVENTION

Now, preferred embodiments of the present invention will be described with reference to the accompanying drawings.

### EXAMPLE 1

### Culture of Mycobacteria and Separation of Genome DNA

Standard strains of mycobacteria were obtained from KCTC (Korean Collection for Type Culture) and ATCC (American Type Culture Collection), clinical strains were obtained from the Korean National Tuberculosis Association, National Masan Tuberculosis Hospital and Pusan National University Hospital. The strains were stored and cultured under the management of clinical microbiology specialist in Pusan National University Hospital. The mycobacteria and pathogens used in the examples are shown in Table 8.

Mycobacteria DNA was extracted by the following processes: Mycobacteria were cultured on Ogawa medium. A loopful of cultured strain was put in eppendorf tube, mixed with 200 μl of InstaGene matrix (Bio-Rad Co.) and incubated at 56° C. for 30 minutes. After 10 minutes' vortex missing, the mixture was placed at 100° C. for 8 minutes. After another 10 minutes' vortex mixing, the mixture was centrifuged at 12,000 rpm for 3 minutes. The supernatant was moved to another tube and stored at −20° C. 2 μl of the DNA solution of each strain was used in the succeeding PCR.

### EXAMPLE 2

### Manufacture of Primers for Amplifying ITS of Mycobacteria

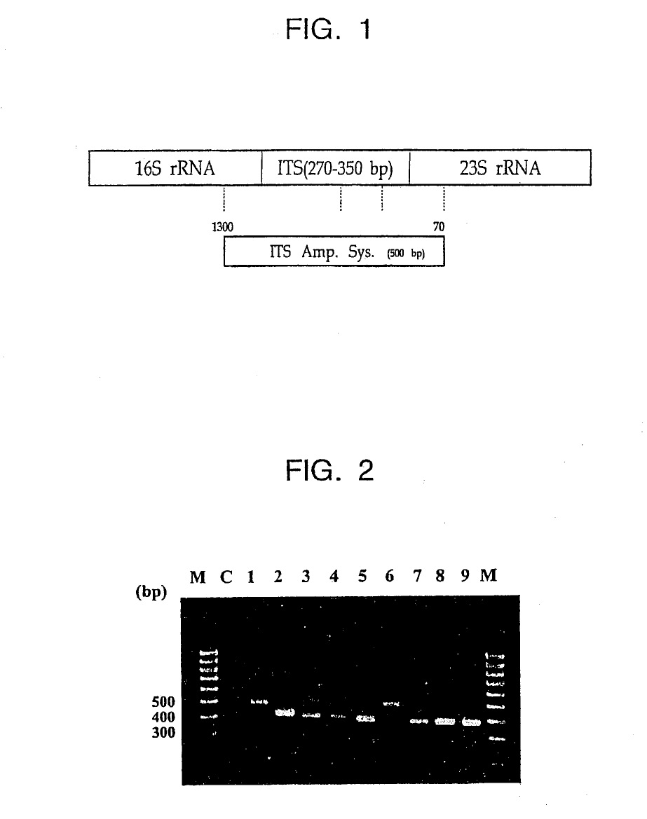

The ITS of mycobacteria and primers used to amplify the ITS by PCR were illustrated in FIG. 1. The primers for amplifying ITS of mycobacteria were constructed from several section of conserved regions of 16S rRNA and 23S rRNA of mycobacteria. DNA sequences of 16S and 23S rRNA of mycobacteria disclosed in GenBank were analyzed with multialignment and blast search. The primers were designed to amplify selectively about 500 bp of ITS region with a part of 16S rRNA and 23S rRNA and have sequences set in forth in SEQ ID NOs: 242 (ITSF) and 243 (ITSR). All the primers used in Examples were manufacture by the concentration of 50 nmol with Perkin-Elmer DNA Synthesizer by BioBasic (Canada).

### EXAMPLE 3

### PCR and Identification of Products

2 μl of the DNA solution obtained in Example 1 was used in PCR. Reaction solution included: 500 mM KCl, 100 mM Tris HCl (pH 9.0), 1% Triton X-100, 0.2 mM dNTP (dATP, dGTP, dTTP and dCTP), 1.5 mM MgCl₂, 1 pmol of primer, 1U of Taq DNA polymerase (Bio Basis Inc.). After denaturated at 94° C. for 5 minutes, the solution was reacted 30 cycles of denaturation at 94° C. for 1 minute, annealing at 60° C. for 1 minute, and elongation at 72° C. for 1 minute. The samples were incubated further for 10 minutes at 72° C. for complete elongation. After the reaction, the PCR products were identified by electrophoresis on 1.5% agarose gel. As expected from the information of GenBank, the ITS amplified using the conserved primers of 16S rRNA and 23S rRNA was about 500 bp.

FIG. 2 is a photograph showing the result of electrophoresis after PCR using several mycobacteria strains and a pair of primers for ITS amplification including a part of 16S rRNA and 23S rRNA of mycobacteria. In this figure, M means molecular weight mark having intervals of 100 bp, C is a control group, lanes 1 to 9 indicate, in turn, M. fortuitum, M chelonae, M. intracellularae, M. avium, M. tuberculosis, M. agri, M. kansasii, M. gordonae, and M. tuberculosis H37Rv. It can be noted that all mycobacteria of lanes 1 to 9 have amplification of ITS gene except control group C.

### EXAMPLE 4

### Determination of DNA Sequence of Mycobacteria ITS

After the identification of PCR products, the reactants of M. fortuitum, M. chelonae, M. abscessus, M. vaccae, M. fiavescens, M. asiaticum, M. porcinum, M. acapulcensis and M. diernhoferi, whose DNA sequences of ITS have not yet been determined, were amplified by PCR. The PCR products were used directly in determining DNA sequence of ITS.

DNA was used by the concentration of 200 μmol. DNA sequence was determined by dye terminator method using universal primer M13 with DNA auto sequencer (Perkin-Elmer ABI prim 377 sequencer).

SEQ ID NOs 1 to 9 indicate DNA sequences of ITS of Mycobacterium fortuitum, Mycobacterium chelonae, Mycobacterium abscessus, Mycobacterium vaccae, Mycobacterium flavescens, Mycobacterium asiaticum, Mycobacterium porcinum, Mycobacterium acapulcensis and Mycobacterium diernhoferi, in turn.

### EXAMPLE 5

### Design and Synthesis of Oligonucleotide Primer

Each primer of about 20 bp was designed from DNA sequences of ITS of Mycobacterium fortuitum, Mycobacterium chelonae, Mycobacterium abscessus, Mycobacterium vaccae, Mycobacterium flavescens, Mycobacterium asiaticum, Mycobacterium porcinum, Mycobacterium acapulcensis and Mycobacterium diernhoferi, obtained in Example 4, and compared with DNA sequences of ITS of other mycobacteria obtained from GenBank by multi-alignment and blast.

Based on the result, conservative DNA sequences of mycobacteria ITS were designed as primers or probes for detection of mycobacteria, and DNA sequences of high polymorphism were designed as species-specific primers or probes. Tables 1 to 7 depict the designed primers or probes with their position and SEQ ID NOs.

TABLE 1

| Target Strain | Probe Name | Position in ITS | SEQ ID NO. |
|---|---|---|---|
| Mycobacteria | MYC1 | Variable to the | 10 |
| | MYC2 | species | 11 |
| | MYC3 | | 12 |
| | MYC4 | | 13 |
| | MYC5 | | 14 |
| TB complex | MTB1 | 166-185 | 15 |
| | MTB2 | 65-84 | 16 |
| | MTB3 | 20-39 | 17 |
| | MTB4 | 41-60 | 18 |
| | MTB5 | 60-79 | 19 |
| | MTB6 | 81-100 | 20 |
| | MTB7 | 125-144 | 21 |
| | MTB8 | 139-158 | 22 |
| | MTB9 | 203-222 | 23 |
| M. avium-M. | MAC1 | 241-260 | 24 |
| intracellularae | MAC2 | 142-161 | 25 |
| (MAC) | MAC3 | 92-111 | 26 |
| | MAC4 | 117-136 | 27 |
| M. fortuitum | FOR1 | 40-59 | 28 |
| | FOR2 | 44-63 | 29 |
| | FOR3 | 64-83 | 30 |
| | FOR4 | 78-97 | 31 |
| | FOR5 | 89-108 | 32 |
| | FOR6 | 109-128 | 33 |
| | FOR7 | 114-133 | 34 |
| | FOR8 | 134-153 | 35 |
| | FOR9 | 157-176 | 36 |
| | FOR10 | 246-265 | 37 |
| | FOR11 | 289-308 | 38 |
| M. chelonae | CHE1 | 11-30 | 39 |
| | CHE2 | 29-48 | 40 |
| | CHE3 | 58-77 | 41 |
| | CHE4 | 78-97 | 42 |
| | CHE5 | 109-128 | 43 |
| | CHE6 | 132-151 | 44 |
| | CHE7 | 171-190 | 45 |
| | CHE8 | 246-265 | 46 |

TABLE 2

| Target Strain | Probe Name | Position in ITS | SEQ ID NO. |
|---|---|---|---|
| M. abscessus | ABC1 | 37-56 | 47 |
| | ABC2 | 55-74 | 48 |
| | ABC3 | 247-266 | 49 |
| | ABC4 | 263-282 | 50 |
| | ABC5 | 270-289 | 51 |
| | ABC6 | 261-280 | 52 |
| M. vaccae | VAC1 | 18-37 | 53 |
| | VAC2 | 38-57 | 54 |
| | VAC3 | 58-77 | 55 |
| | VAC4 | 118-137 | 56 |
| | VAC5 | 138-157 | 57 |
| | VAC6 | 158-177 | 58 |
| | VAC7 | 178-197 | 59 |
| | VAC8 | 199-218 | 60 |
| | VAC9 | 219-238 | 61 |
| | VAC10 | 265-284 | 62 |
| | VAC11 | 298-317 | 63 |
| | VAC12 | 321-340 | 64 |
| M. flavescens | FLA1 | 12-31 | 65 |
| | FLA2 | 32-51 | 66 |
| | FLA3 | 52-71 | 67 |
| | FLA4 | 72-91 | 68 |
| | FLA5 | 105-124 | 69 |
| | FLA6 | 125-144 | 70 |
| | FLA7 | 173-192 | 71 |
| | FLA8 | 278-297 | 72 |
| M. gordonae | GOR1 | 84-103 | 73 |
| | GOR2 | 249-268 | 74 |
| | GOR3 | 216-235 | 75 |
| | GOR4 | 201-220 | 76 |
| | GOR5 | 223-242 | 77 |

TABLE 3

| Target Strain | Probe Name | Position in ITS | SEQ ID NO. |
|---|---|---|---|
| M. terrae | TER1 | 178-197 | 78 |
| | TER2 | 237-256 | 79 |
| | TER3 | 24-43 | 80 |
| | TER4 | 70-89 | 81 |
| | TER5 | 89-108 | 82 |
| | TER6 | 102-121 | 83 |
| | TER7 | 122-141 | 84 |
| | TER8 | 142-161 | 85 |
| | TER9 | 162-181 | 86 |
| | TER10 | 182-201 | 87 |
| | TER11 | 202-221 | 88 |
| | TER12 | 222-241 | 89 |
| | TER13 | 238-257 | 90 |
| | TER14 | 307-326 | 91 |
| | TER15 | 322-341 | 92 |
| | TER16 | 342-361 | 93 |
| | TER17 | 362-381 | 94 |
| | TER18 | 382-401 | 95 |
| | TER19 | 12-21 | 96 |
| | TER20 | 31-50 | 97 |
| | TER21 | 52-71 | 98 |
| | TER22 | 72-91 | 99 |
| | TER23 | 82-101 | 100 |
| M. scrofulaceum | SCO1 | 118-137 | 101 |
| | SCO2 | 131-150 | 102 |
| | SCO3 | 210-229 | 103 |
| | SCO4 | 84-103 | 104 |
| | SCO5 | 152-171 | 105 |
| | SCO6 | 200-219 | 106 |
| | SCO7 | 221-240 | 107 |
| | SCO8 | 241-260 | 108 |
| M. kansasii | KAN1 | 35-54 | 109 |
| | KAN2 | 238-257 | 110 |
| | KAN3 | 83-102 | 111 |
| | KAN4 | 214-233 | 112 |

TABLE 4

| Target Strain | Probe Name | Position in ITS | SEQ ID NO. |
|---|---|---|---|
| M. szulgai | SZU1 | 124-143 | 113 |
| | SZU2 | 209-228 | 114 |
| | SZU3 | 227-246 | 115 |
| | SZU4 | 247-166 | 116 |
| M. marinum and | MAR-ULC1 | 85-104 | 117 |
| M ulcerans | MAR-ULC2 | 128-147 | 118 |
| | MAR-ULC3 | 224-243 | 119 |
| M. gastri | GAS1 | 85-104 | 120 |
| | GAS2 | 145-164 | 121 |
| | GAS3 | 133-152 | 122 |
| | GAS4 | 239-258 | 123 |
| M. xenopi | XEN1 | 190-209 | 124 |
| | XEN2 | 1-20 | 125 |
| | XEN3 | 21-40 | 126 |
| | XEN4 | 41-60 | 127 |
| | XEN5 | 61-80 | 128 |
| | XEN6 | 81-100 | 129 |
| | XEN7 | 121-140 | 130 |
| | XEN8 | 141-160 | 131 |
| | XEN9 | 201-220 | 132 |
| | XEN10 | 221-240 | 133 |
| M. genavense | GEN1 | 190-209 | 134 |
| | GEN2 | 85-104 | 135 |
| | GEN3 | 131-150 | 136 |
| | GEN4 | 147-166 | 137 |
| | GEN5 | 186-205 | 138 |
| | GEN6 | 206-225 | 139 |
| | GEN7 | 226-245 | 140 |
| | GEN8 | 240-265 | 141 |
| M. malmoense | MAL1 | 203-222 | 142 |
| | MAL2 | 29-48 | 143 |
| | MAL3 | 136-155 | 144 |
| | MAL4 | 222-241 | 145 |
| | MAL5 | 242-261 | 146 |
| M. simiae | SIM1 | 83-102 | 147 |
| | SIM2 | 129-148 | 148 |
| | SIM3 | 209-227 | 149 |
| | SIM4 | 22-41 | 150 |
| | SIM5 | 80-99 | 151 |
| | SIM6 | 136-155 | 152 |
| | SIM7 | 241-260 | 153 |

TABLE 5

| Target Strain | Probe Name | Position in ITS | SEQ ID NO. |
|---|---|---|---|
| M. smegmatis | SMEG1 | 17-36 | 154 |
| | SMEG2 | 37-56 | 155 |
| | SMEG3 | 57-76 | 156 |
| | SMEG4 | 77-96 | 157 |
| | SMEG5 | 97-116 | 158 |
| | SMEG6 | 117-136 | 159 |
| | SMEG7 | 137-156 | 160 |
| | SMEG8 | 157-176 | 161 |
| | SMEG9 | 177-196 | 162 |
| | SMEG10 | 193-212 | 163 |
| | SMEG11 | 60-80 | 164 |
| | SMEG12 | 112-131 | 165 |
| M. shimoidei | SHI1 | 89-108 | 166 |
| | SHI2 | 20-39 | 167 |
| | SHI3 | 70-89 | 168 |
| | SHI4 | 97-116 | 169 |
| | SHI5 | 135-154 | 170 |
| | SHI6 | 224-243 | 171 |
| | SHI7 | 244-263 | 172 |
| M. habana | HAB1 | 86-105 | 173 |
| | HAB2 | 17-36 | 174 |
| | HAB3 | 51-70 | 175 |
| | HAB4 | 81-100 | 176 |
| | HAB5 | 134-153 | 177 |
| | HAB6 | 175-194 | 178 |
| | HAB7 | 200-219 | 179 |
| | HAB8 | 242-261 | 180 |
| M. farcinogen | FAR1 | 122-141 | 181 |
| | FAR2 | 111-130 | 182 |
| | FAR3 | 22-41 | 183 |
| | FAR4 | 48-67 | 184 |
| | FAR5 | 76-95 | 185 |
| | FAR6 | 108-127 | 186 |
| | FAR7 | 114-133 | 187 |
| | FAR8 | 275-294 | 188 |
| | FAR9 | 295-314 | 189 |

TABLE 6

| Target Strain | Probe Name | Position in ITS | SEQ ID NO. |
|---|---|---|---|
| M. asiaticum | ASI1 | 82-101 | 190 |
| | ASI2 | 145-164 | 191 |
| | ASI3 | 189-208 | 192 |
| | ASI4 | 274-293 | 193 |
| M. porcinum | POR1 | 45-64 | 194 |
| | POR2 | 13-32 | 195 |
| | POR3 | 67-86 | 196 |
| | POR4 | 91-110 | 197 |
| | POR5 | 115-134 | 198 |
| | POR6 | 137-156 | 199 |
| | POR7 | 164-183 | 200 |
| | POR8 | 194-213 | 201 |
| | POR9 | 221-240 | 202 |
| | POR10 | 273-292 | 203 |
| | POR11 | 298-317 | 204 |
| | POR12 | 325-344 | 205 |
| M. acapulcensis | ACA1 | 66-85 | 206 |
| | ACA2 | 112-131 | 207 |
| | ACA3 | 132-151 | 208 |
| | ACA4 | 178-197 | 209 |
| | ACA5 | 198-217 | 210 |
| | ACA6 | 219-238 | 211 |
| | ACA7 | 242-261 | 212 |
| | ACA8 | 262-281 | 213 |
| | ACA9 | 318-337 | 214 |
| | ACA10 | 350-369 | 215 |
| M. diernhoferi | DIE1 | 16-35 | 216 |
| | DIE2 | 36-55 | 217 |
| | DIE3 | 62-81 | 218 |
| | DIE4 | 103-122 | 219 |
| | DIE5 | 154-173 | 220 |
| | DIE6 | 175-194 | 221 |
| | DIE7 | 195-214 | 222 |
| | DIE8 | 232-251 | 223 |
| | DIE9 | 261-280 | 224 |
| | DIE10 | 282-301 | 225 |
| | DIE11 | 304-343 | 226 |
| | DIE12 | 344-363 | 227 |

TABLE 7

| Target Strain | Probe Name | Position in ITS | SEQ ID NO. |
|---|---|---|---|
| M. para-tuberculosis | PARA1 | 7-26 | 228 |
| | PARA2 | 30-49 | 229 |
| | PARA3 | 40-59 | 230 |
| | PARA4 | 50-59 | 231 |
| | PARA5 | 71-90 | 232 |
| | PARA6 | 83-102 | 233 |
| | PARA7 | 103-122 | 234 |
| | PARA8 | 135-154 | 235 |
| | PARA9 | 157-176 | 236 |
| | PARA10 | 178-197 | 237 |
| | PARA11 | 198-217 | 238 |
| | PARA12 | 219-238 | 239 |
| | PARA13 | 241-260 | 240 |
| M. sp | SP1 | 225-244 | 241 |

### EXAMPLE 6

### Result of PCR using Primers for detecting Mycobacteria

Genus-specific primers, designed from conserved DNA sequence in mycobacteria, were used for detecting mycobacteria. Among the primers manufactured from the ITS sequence of 270-350 bp, a pair of primers, ITSF (SEQ ID NO. 242) and MYC2 (SEQ ID NO. 11) were used to proceed PCR. As a result, amplified nucleotides of about 350 bp were obtained in mycobacteria strains, while no amplification was occurred in Staphylococcus aureus, Enterococcus faecium and Serratia marcescens. Therefore, it is understood that the primers could be used for detecting mycobacteria.

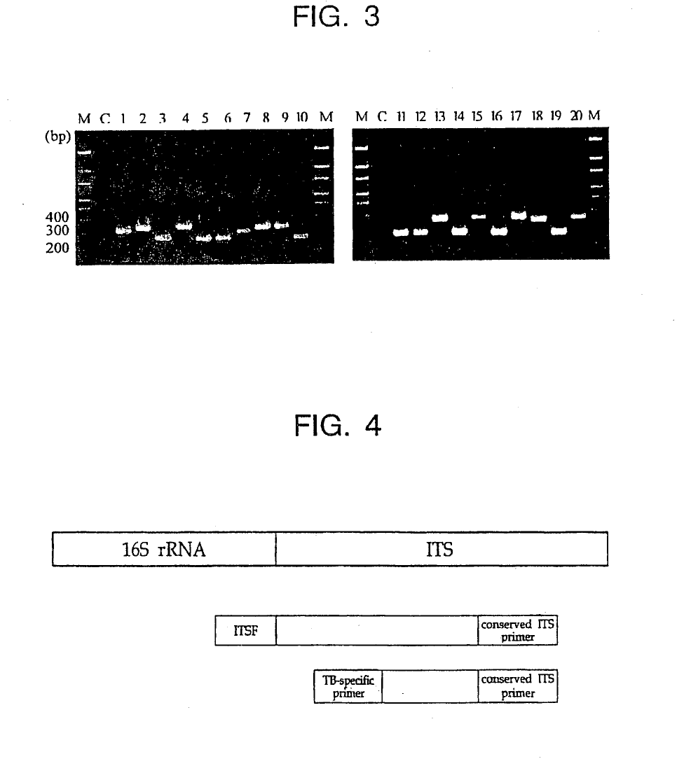

FIG. 3 is a photograph showing the result of electrophoresis after PCR using several mycobacteria strains and a pair of primers (ITSF and MYC2) for detecting mycobacteria. In this figure, M indicates a size marker of 100 bp ladder; C indicates a negative control; lane 1 indicates M. abscessus ATCC 19977; lane 2, M. agri ATCC 27406; lane 3, M. asiaticum ATCC 25276; lane 4, M. austroafricanum ATCC 33464; lane 5, M. avium ATCC 25291; lane 6, M. bovis ATCC 19210; lane 7, M. chelonae ATCC 35752; lane 8, M. flavescens ATCC 14474; lane 9, M. fortuitum ATCC 6841; lane 10, M. gordonae ATCC 14470; lane 11, M. intracellularae ATCC 13950; lane 12, M. kansasii ATCC 12478; lane 13, M. phlei ATCC 354; lane 14, M. scrofulaceum ATCC 19981; lane 15, M. smegmatis ATCC 21701; lane 16, M. szulgai ATCC 35799; lane 17, M. terrae ATCC 15755; lane 18, M. triviale ATCC 23292; lane 19, M. tuberculosis H37Rv; and lane 20, M. vaccae ATCC 15483. It can be seen that all mycobacteria of lanes 1 to 20 show ITS amplification except negative control C.

Table 8 shows the results of PCR using a pair of primers (ITSF and MYC2) for detecting mycobacteria. In this table, +indicates amplification occurred and −indicates no amplification. It can be seen that no amplification has occurred in order strains than mycobacteria.

TABLE 8

| Name of Strain | result | Name of Strain | result |
|---|---|---|---|
| M. tuberculosis H37Rv | + | M. Phlei ATCC 354 | + |
| M. bovis ATCC 19210 | + | Aeromonas hydrophila | − |
| M. avium ATCC 25291 | + | Burkholderia cepacia | − |
| M intracellulare ATCC 13950 | + | Candida albicans | − |
| M. abscessus ATCC 19977 | + | Citrobacter freundii | − |
| M. chelonae ATCC 35752 | + | Enterobacter aerogenes | − |
| M. flavescens ATCC 14474 | + | Enterobacter cloacae | − |
| M. fortuitum ATCC 6841 | + | Enterobacter faecalis | − |
| M. gastri ATCC 15754 | + | Enterobacter faecium | − |
| M. genavense ATCC 51233 | + | Enterobacter raffinosis | − |
| M. gordonae ATCC 14470 | + | Escherichia coli | − |
| M. kansasii ATCC 12478 | + | Klebsiella pneumoniae | − |
| M. malmoense ATCC 29571 | + | Plesiomonas shigelloides | − |
| M. scrofulaceum ATCC | + | Proteus mirabilis | − |
| 19981 | | | |
| M. simiae ATCC 25275 | + | Proteus vulgaris | − |
| M. smegmatis ATCC 21701 | + | Providencia rettgeri | − |
| M. szulgai ATCC 35799 | + | Pseudomonas aeruginosa | − |
| M. terrae ATCC 15755 | + | Rohnella aquatilis | − |
| M. vaccae ATCC 15483 | + | Salmonella spp. | − |
| M. xenopi ATCC 19250 | + | Serratia marcescens | − |
| M. marinum ATCC 927 | + | Shewanella putrefaciens | − |
| M. ulcerance ATCC 19423 | + | Shigella flexneri | − |
| M. porcinum ATCC 33776 | + | Shigella sonnei | − |
| M. asiaticum ATCC 25276 | + | Staphylococcus epidermidis | − |
| M. acapulcensis ATCC 14473 | + | Staphylococcus aureus | − |
| M. diernhoferi ATCC 19340 | + | Streptococcus agalactiae | − |

| | | | |
|---|---|---|---|
| M. agri ATCC 27406 | + | Streptococcus intermidius | − |
| M. austroafricanum ATCC | + | Streptococcus pneumoniae | − |
| 33464 | | | |
| M. triviale ATCC 23292 | + | Vibrio parahemolyticus | − |

### EXAMPLE 7

### Result of PCR using Primers for TB Complex

Test for identifying each strain using the oligonucleotide primers manufactured in the previous Examples was confirmed by amplification in PCR. For identifying TB complex, multiplex PCR was carried using a pair of primers (ITSF and MYC2) for detecting mycobacteria and a pair of primers (MTB 2 and MYC 2; SEQ ID Nos. 16 and 11) for distinguishing TB complex from NTM.

FIG. 4 is a schematic diagram of multiplex PCR for detecting mycobacteria and simultaneously distinguishing TB complex from NTM.

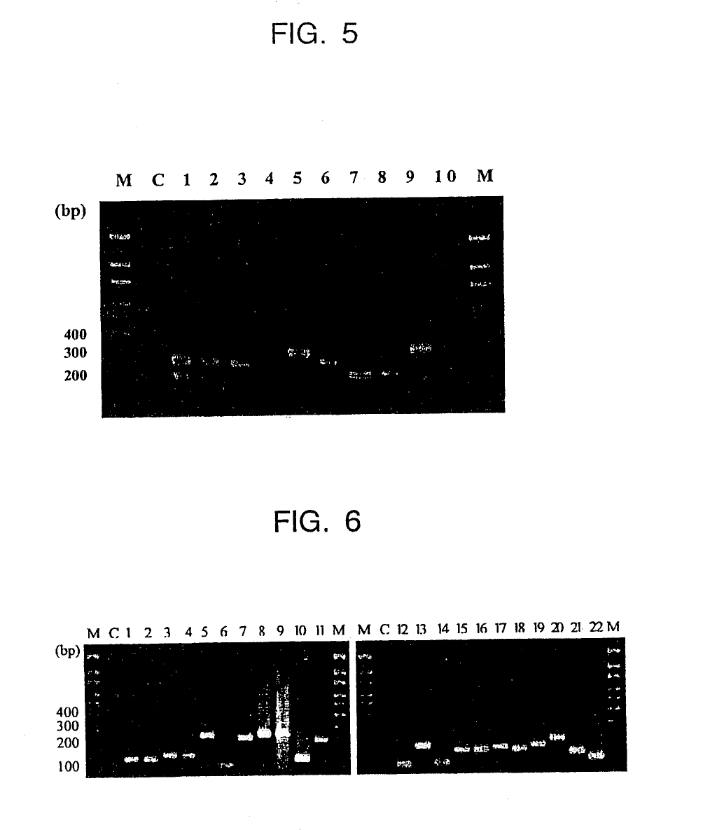

FIG. 5 is a photograph showing the result of electrophoresis after multiplex PCR using a pair of primers (ITSF and MYC2) for detecting mycobacteria and a pair of primers (MTB2 and MYC2) for distinguishing TB complex from NTM. In this figure, M indicates a size marker of 100 bp ladder; C indicates a negative control; lanes 1 and 2 indicate TB complex, M. tuberculosis H37Rv and M. bovis, respectively; and lanes 3 to 10 indicate M. avium, M. intracellularae, M. fortuitum, M. chelonae, M. gordonae, M. szulgai, M. terrae, and M. scrofulaceum ATCC 19981, in turn. In lanes 1 and 2 of TB complex, two bands are formed by double amplification due to primers for detecting mycobacteria and primers for TB complex, while in the order lanes of NTM strains, only one band is formed, which confirms single amplification.

### EXAMPLE 8

### Result of PCR using Primers for Identifying NTM Strains

PCR was carried out using species-specific primers manufacture from DNA sequence of polymorphic site of each strain for identifying NTM species.

Specifically, after selecting SEQ ID NOs. 16 and 21 for M. tuberculosis H37Rv and M. bovis, SEC ID NOs: 24 to 27 for M. avium and M. Intracellularae, SEQ ID Nos. 29 and 37 for M. fortuitum, SEQ ID Nos. 41 and 44 for M. chelonae, SEQ ID Nos. 48 and 49 for M. abscessus, SEQ ID Nos. 55 and 63 for M. vaccac, SEQ ID Nos. 66 and 72 for M. flavescens, SEQ ID Nos. 73 and 75 for M. gordonae, SEQ ID Nos. 88 and 96 for M. terrae, SEQ ID Nos. 102 and 103 for M. scrofulaceum, SEQ ID Nos. 109 and 110 for M. kansasii, SEQ ID Nos. 113 and 114 for M. szulgai, SEQ ID Nos. 117 and 119 for M. marinum and M. ulcerans, SEQ ID Nos. 120 and 123 for M. gastri, SEQ ID Nos. 128 and 132 for M. xenopi, SEQ ID Nos. 135 and 141 for M. genavense, SEQ ID Nos. 143 and 145 for M. malmoense, SEQ ID Nos. 147 and 149 for M. simiae, and SEQ ID Nos. 154 and 159 for M. smegmatis, each mycobacteria was carried out PCR using each pair of primers of which the first has been sense strand and the second has been antisense strand. After the reaction, each resultant was treated by electrophoresis.

FIG. 6 is a photograph showing the result of electrophoresis after PCR using each mycobacteria and each pair of species-specific primers designed from nucleotide sequence of polymorphic region of each NTM. In this figure, M indicates a size marker of 100 bp ladder; C indicates a negative control; lanes 1 and 2 indicate TB complex, M. tuberculosis H37Rv and M. bovis ATCC 19210, respectively; and lanes 3 and 4 indicate M. avium ATCC 25291 and M. intracellularae ATCC 13950, respectively; lane 5 indicates M. fortuitum ATCC 6841; lane 6, M. chelonae ATCC 35752; lane 7, M. abscessus ATCC 19977; lane 8, M. vaccae ATCC 15483; lane 9, M. flavescens ATCC 14474; lane 10, M. gordonae ATCC 14470; lane 11, M. terrae ATCC 15755; lane 12, M. scrofulaceum ATCC 19981; lane 13, M. kansasii ATCC 12478; lane 14, M. szulgai ATCC 35799; lane 15, M. marinum ATCC 927; lane 16, M. ulcerans ATCC 19423; lane 17, M. gastri ATCC 15754; lane 18, M. xenopi ATCC 19250; lane 19, M. genavense ATCC 1233; lane 20, M. malmoense ATCC 29571; lane 21, M. simiae ATCC 25275; and lane 22 indicates M. smegmatis ATCC 21701. Species-specific amplifications can be seen in lanes 1 to 22.

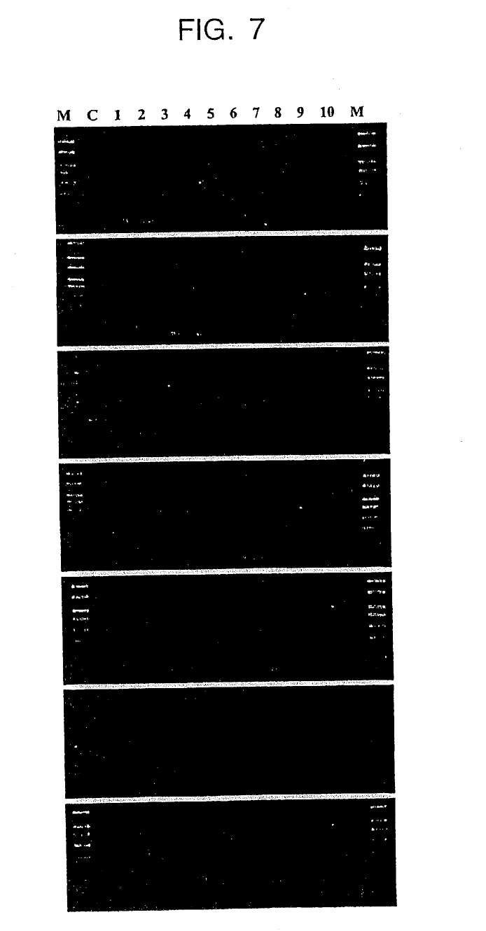

FIG. 7 is a photograph showing the result of electrophoresis after PCR using several mycobacteria and each pair of species-specific primers designed from nucleotide sequence of polymorphic region of each NTM. In this figure, the first photograph using the pair of primers (ITSF and MYC2) specific for TB complex shows amplification in lanes 1 and 2 of M. tuberculosis H37Rv and M. bovis; the second using the pair of primers (MAC1 and MAC4) specific for M. avium and M. intracellularae shows amplifications in lanes 3 and 4 of M. avium and M. intracellularae; the third using the pair of primers (FOR2 and FOR10) specific for M. fortuitum shows amplification in lane 5 of M. fortuitum; the fourth using the pair of primers (CHE3 and CHE6) specific for M. chelonae shows amplification in lane 6 of M. chelonae; the fifth using the pair of primers (GOR1 and GOR2) specific for M. gordonae shows amplification in lane 7 of M. gordonae; the sixth using the pair of primers (SZU1 and SZU2) specific for M. szulgai shows amplification in lane 8 of M. szulgai; and the seventh using the pair of primers (SCO1 and SCO2) specific M. scrofulaceum for shows amplification in lane 10 of M. scrofulaceum. Lane 9 indicates M. terrae, which shows no amplification with the above species-specific primer pairs.

Therefore, PCR using each pair of species-specific primers can detect and identify specifically each species of mycobacteria.

As described above, identifying DNA sequences of ITS (Internal Transcribed Spacer region) of non-tuberculosis mycobacteria, Mycobacterium fortuitum, Mycobacterium chelonae, Mycobacterium abscessus, Mycobacterium vaccae, Mycobacterium flavescens, Mycobacterium asiaticum, Mycobacterium porcinum, Mycobacterium acapulcensis and Mycobacterium diernhoferi, and using the DNA sequences, oligonucleotide primers or probes can been designed for detecting and identifying mycobacteria. Using the primers for PCR or probes for hybridization, it is possible to detect mycobacteria, distinguish TB complex from NTM and identify mycobacteria species with rapidity and effectiveness. Such detection and identification method makes the diagnosis of complex infection effective, and therefore, it is possible to treat accurately mycobacterial infection including tuberculosis.

While this invention has been described in connection with what is presently considered to be the most practical and preferred embodiments, it is to be understood that the invention is not limited to the disclosed embodiment, but, on the contrary, it is intended to cover various modifications and equivalent arrangements included with the spirit and scope of the appended claims.

What is claimed is:

1. An oligonucleotide for detection and identification of MAC (Mycobacterium avium and Mycobacterium intracellulare) set forth in one of SEQ ID NOs: 24 to 27.
